(19) United States
(12) Patent Application Publication
(10) Pub. No.: US 2026/0179739 A1
(43) Pub. Date: Jun. 25, 2026

(54) AI Method to Associate Proprietary Coding and Patient Data with Standards

(71) Applicant: Oracle International Corporation, Redwood Shores, CA (US)

(72) Inventors: Amitabh Saikia, Mountain View, CA (US); Raefer Christopher Gabriel, San Mateo, CA (US); Suhas Srinivas Uliyar, Woodinville, WA (US); Amy Marie Willson, East Aurora, NY (US); Vadim Khotilovich, Leawood, KS (US); Travis Ray Bean, Shawnee, KS (US); Natalee Agassi, Blue Bell, PA (US)

(73) Assignee: Oracle International Corporation, Redwood Shores, CA (US)

(21) Appl. No.: 19/381,436

(22) Filed: Nov. 6, 2025

Related U.S. Application Data
(60) Provisional application No. 63/736,107, filed on Dec. 19, 2024.

(51) Int. Cl. G16H 10/60

(52) U.S. Cl. CPC G16H 10/60

(57) ABSTRACT

Techniques for mapping proprietary codes with standard codes based on a similarity between network relationships associated with the respective standard codes and proprietary codes are disclosed. The system generates a cross-domain network having sets of terminology, including at least a set of proprietary codes and a set of standard codes. The network includes (a) a plurality of nodes that represent terms in the sets of terminology, (b) inter-terminology connections between nodes across sets of terminology, and (c) intra-terminology connections between nodes within sets of terminology. The system identifies relationships for the terms that define the connections between the nodes. The system generates vector embeddings for the terms by applying a vector embedding function to the relationships and/or the terms associated with each term. Similarity measures are calculated for vector embedding pairs. Pairs having a similarity measure that exceeds a threshold are identified as semantic matches.

### BENEFIT CLAIMS; RELATED APPLICATIONS; INCORPORATION BY REFERENCE

This application claims the benefit of U.S. Provisional Patent Application 63/736,107, filed Dec. 19, 2024, that is hereby incorporated by reference.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to computer-implemented systems and methods for mapping proprietary codes to standard codes. In particular, the present disclosure relates to machine learning-based similarity computation techniques that evaluate relationships between proprietary code datasets and standard code datasets to generate code mappings in a computing environment.

### BACKGROUND

Healthcare data present across multiple healthcare systems became connected and more accessible with the widespread adoption of Electronic Health Records (EHRs) by healthcare providers. EHRs have become an integral part of modern healthcare systems, offering several benefits over traditional paper-based records. EHRs are digital versions of a patient's medical history, including their diagnosis, treatments, medications, allergies, laboratory results, and other relevant healthcare information. This information may be presented as code values that are present under their respective type of field tables, known as code sets. A code set has a list of code values used to describe a specific purpose/intent. Healthcare data across various client domains are filled with ambiguous textual representations that may be present in the form of synonyms, acronyms, and abbreviations. The ambiguous textual representations create a large variance, for although the code values have semantic equivalence, the various code sets are named differently.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIG. 2 illustrates an example set of operations for mapping proprietary codes with standard codes using similarity between network relationships in accordance with one or more embodiments;

FIG. 3 illustrates an example embodiment of a proprietary code/standard code mapping within a cross-terminology network;

FIG. 4A illustrates a machine learning (ML) engine in accordance with one or more embodiments;

FIG. 4B illustrates an example set of operations of an ML engine in accordance with one or more embodiments; and

FIG. 5 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. CROSS-TERMINOLOGY MAPPING SYSTEM ARCHITECTURE
3. MAPPING PROPRIETARY CODES WITH STANDARD CODES USING SIMILARITY BETWEEN NETWORK RELATIONSHIPS
4. EXAMPLE CROSS-TERMINOLOGY NETWORK MAPPING
5. MACHINE LEARNING ARCHITECTURE
6. MACHINE LEARNING ENGINE OPERATIONS
7. GENERATIVE AI MODELS
8. PRACTICAL APPLICATIONS, ADVANTAGES, AND IMPROVEMENTS
9. HARDWARE OVERVIEW
10. MISCELLANEOUS; EXTENSIONS

### 1. General Overview

One or more embodiments map proprietary codes with standard codes based on a similarity between network relationships associated with the respective standard codes and proprietary codes. Proprietary codes, as referred to herein, include reference codes particular to organizations or vendors. Standard codes, as referred to herein, are industry or standardized codes (e.g., Systematized Nomenclature of Medicine—Clinical Terms (SNOMED CT), Logical Observation Identifiers Names and Codes (LOINC), RxNorm). Mapping proprietary codes to standard codes enhances data interoperability and plays a crucial role in improving the overall quality of healthcare delivery and patient outcomes.

Initially, the system generates a cross-domain network. The cross-domain network includes sets of terminology, including at least a set of proprietary codes and a set of standard codes. The cross-domain network includes (a) a plurality of nodes that represent terms in the sets of terminology, (b) inter-terminology connections between nodes across sets of terminology, and (c) intra-terminology connections between nodes within sets of terminology. The system identifies relationships for terms in the set of proprietary codes and relationships for terms in the set of standard codes. The relationships define the connections between the nodes.

In one or more embodiments, the system generates vector embeddings for the terms of the standard codes and the terms of the proprietary codes. The system generates the vector embeddings by applying a vector embedding function to the relationships and/or the terms associated with each term in the set of standard codes and the set of proprietary codes.

In one or more embodiments, the system compares vector embedding for each term of the proprietary codes to the vector embeddings for each term of the standard codes. Proprietary code and standard code term pairs with similarity measures above a threshold are identified as semantic matches. The system stores an association, or mapping, between the proprietary code and the standard code that are identified as semantic matches.

In one or more embodiments, a reasoner is applied to the terms of the terminologies to identify relationships between the terms within a terminology and between terms across terminologies.

One or more embodiments described in this Specification and/or recited in the claims may not be included in this General Overview section.

### 2. Cross-Terminology Mapping System Architecture

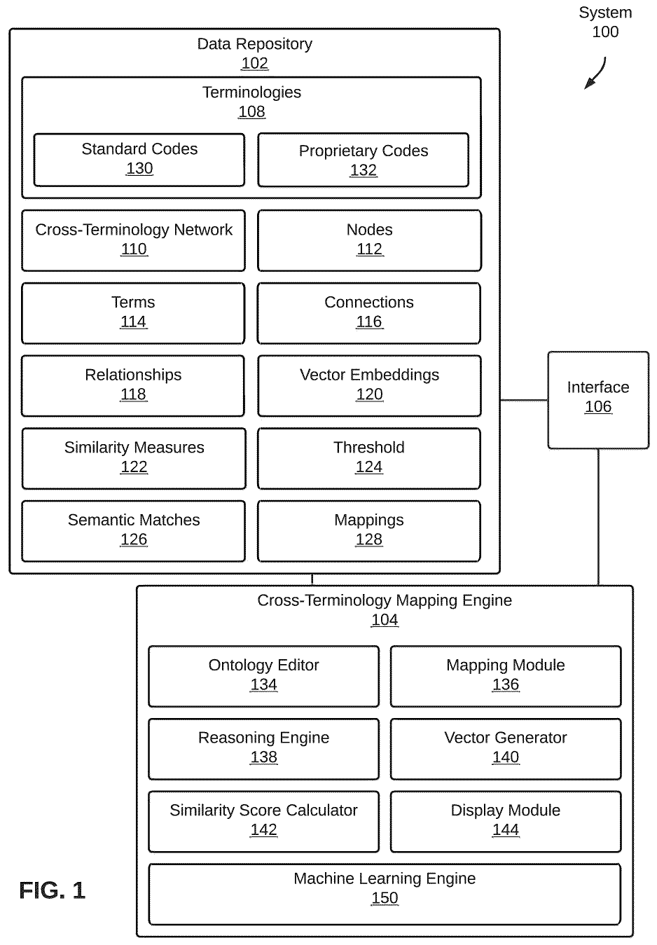

FIG. 1 illustrates a system 100 in accordance with one or more embodiments. As illustrated in FIG. 1, the system 100 includes a data repository 102, a cross-terminology mapping engine 104, and an interface 106. The system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, data repository 102 is any type of storage unit and/or device (e.g., a file system, database, collection of tables, or any other storage mechanism) for storing data. Furthermore, data repository 102 may include multiple different storage units and/or devices. The multiple different storage units and/or devices may or may not be of the same type or located at the same physical site. Furthermore, data repository 102 may be implemented or executed on the same computing system as the cross-terminology mapping engine 104 and the interface 106. Additionally, or alternatively, data repository 102 may be implemented or executed on a computing system separate from the cross-terminology mapping engine 104 and the interface 106. Data repository 102 may be communicatively coupled to the cross-terminology mapping engine 104 and the interface 106 via a direct connection or via a network.

In one or more embodiments, data repository 102 is populated with information from a variety of sources and/or systems. Data repository 102 may be populated with numerous data, such as terminologies 108, cross-terminology network 110, nodes 112, terms 114, connections 116, relationships 118, vector embeddings 120, similarity measures 122, threshold 124, semantic matches 126, and mappings 128. Information describing mapping proprietary codes with standard codes using similarity between network relationships may be implemented across any of components within the system 100. However, this information is illustrated within the data repository 102 for purposes of clarity and explanation.

In one or more embodiments, terminologies 108 are used to ensure consistent communication, interoperability, and accurate documentation across healthcare systems. Terminologies 108 include standard codes 130 and proprietary codes 132. Standard codes 130 are a set of industry or standardized codes that are widely adopted and used across the healthcare industry. Standard codes 130 represent various aspects of patient care, procedures, diagnoses, medications, and other healthcare-related information. Proprietary codes 132 are reference codes for clinical and/or non-clinical events or entities that are customized for consumers. When creating proprietary codes 132, local practice may be favored over uniformity of content, resulting in different consumers having unique sets of proprietary codes 132. Although the names of the proprietary codes 132 may differ between consumers, many proprietary codes 132 have semantic equivalences.

In one or more embodiments, standard codes 130 include International Classification of Diseases (ICD), SNOMED CT, LOINC, Current Procedural Terminology (CPT), RxNorm, North American Nursing Diagnosis Association International (NANDA-I), Healthcare Common Procedure Coding System (HCPCS), Diagnostic and Statistical Manual of Mental Disorders (DSM), and Anatomical Therapeutic Chemical Classification System (ATC). ICD, maintained by the World Health Organization (WHO), is for coding diseases, signs, symptoms, and procedures. SNOMED CT is a comprehensive, multilingual terminology for clinical documentation used to encode clinical concepts like diagnoses, symptoms, and procedures. SNOMED CT supports electronic health records (EHRs) with over 300,000 clinical terms. LOINC standardize laboratory and clinical observations are used widely for lab results, clinical measurements, and other observations in electronic systems. CPT, maintained by the American Medical Association, includes standard codes for medical, surgical, and diagnostic procedures. RxNorm is a normalized naming system for generic and branded drugs maintained by the U.S. National Library of Medicine (NLM). RxNorm supports EHR systems in managing medication data and prescriptions. NANDA-I provides standard nursing diagnoses. HCPCS are codes for products, supplies, and services not included in CPT and is maintained by the Centers for Medicare & Medicaid Services (CMS). DSM is a classification of mental health disorders maintained by the American Psychiatric Association (APA) and used by clinicians and researchers to diagnose mental health conditions. OMAHA System is comprehensive terminology for documenting nursing practice and community health. ATC is maintained by the WHO and classifies drugs based on their therapeutic use and chemical properties.

In one or more embodiments, standard codes 130 include Unified Medical Language System (UMLS), Medical Dictionary for Regulatory Activities (MedDRA), International Classification of Functioning, Disability, and Health (ICF), International Union of Pure and Applied Chemistry (IUPAC), National Drug Code (NDC), Procedure Coding System (PCS), and Digital Imaging and Communications in Medicine (DICOM). UMLS, maintained by NLM, integrates various medical terminologies into a single framework for better data exchange. UMLS aides in mapping terms across different coding systems like ICD, SNOMED CT, and LOINC. MedDRA is standard terminology for adverse event reporting in clinical trials and pharmacovigilance maintained by the International Council for Harmonisation. ICF describes health conditions in terms of functioning and disability and is maintained by the WHO. MedDRA is used in rehabilitation, disability evaluation, and social care services. IUPAC Nomenclature provides chemical naming standards, including those for drug molecules. NDC, maintained by the U.S. Food and Drug Administration, identifies drugs in the United States. PCS, maintained by CMS, codes procedures performed in hospital settings (used alongside ICD). DICOM standardizes medical imaging data formats and communication protocols. DICOM is used in radiology and imaging systems to exchange and store images like MRIs, CT scans, and X-rays.

In one or more embodiments, proprietary codes 132 include Medi-Span, First Databank (FDB), Truven Micromedex, and UpToDate. Medi-Span, owned by Wolters Kluwer Health, includes drug databases with information on drug interactions, dosage, and safety. FDB, owned by Hearst Health, provides clinical drug knowledge and decision support. Truven Micromedex, owned by IBM Watson Health, formerly Truven Health Analytics, provides evidence-based drug, disease, and toxicology information. UpToDate, owned by Wolters Kluwer Health, provides clinical decision support resource offering evidence-based guidelines.

In one or more embodiments, proprietary codes 132 include Code Set 72. Code Set 72, also known as Cerner Clinical Event Codes, is a proprietary code set maintained by Cerner Corporation. Code Set 72 is an extensive collection of codes used to represent various clinical and non-clinical events, including clinical documents, note types, immunizations, and clinical observations, such as laboratory results and vital signs. Code Set 72 is highly customized by Cerner clients, and the specific codes used may vary depending on the client's healthcare system. The general structure and purpose of the code set remain consistent across Cerner clients. Code Set 72 is a very large code set, encompassing a wide range of clinical events. The specific codes used in Code Set 72 are tailored to meet the specific needs of each Cerner client.

In one or more embodiments, proprietary codes 132 include Multum, a database that offers detailed drug information to support clinical decision-making. Multum is widely integrated into systems, like EHRs and consumer drug resources. Multum provides critical data for medications, including drug interactions, dosages, and therapeutic uses, aimed at promoting safe medication practices. Managed by Oracle Health (formerly Cerner), Multum offers several tools tailored to healthcare organizations, including Lexicon Plus, which supplies comprehensive drug and disease nomenclature, and VantageRx, which delivers drug knowledge through a structured database format. The database is frequently used in clinical environments to improve prescribing accuracy and avoid adverse drug events by highlighting interactions and warnings. Multum data supports software development kits (SDKs), e.g., addVantageRx and SubscribeRx, which allow organizations to embed or integrate drug content directly into their applications. This makes it an essential component in both consumer-oriented drug information portals and provider-based medication management systems.

In one or more embodiments, cross-terminology network 110 is an ontological structure or framework that connects and maps concepts across multiple terminologies. Cross-terminology network 110 allows for seamless translation and interoperability between different coding systems used in healthcare. Cross-terminology network 110 forms a web of linked nodes from various terminologies, facilitating efficient information sharing and retrieval across platforms that use different coding systems.

In one or more embodiments, nodes 112 represent individual entities or concepts (e.g., conditions, medications, procedures, or patient data elements) within cross-terminology network 110. Each node serves as a point of reference or a unique data unit labeled with a term, i.e., a diagnosis code or medication name, and nodes are interconnected through relationships to form a structured web of healthcare knowledge.

In one or more embodiments, terms 114 are standardized labels used to describe specific concepts within a set of terminology, e.g., diseases, medications, procedures, or symptoms. Each term corresponds to a unique code in a set of terminologies and ensures consistency and precision in data documentation and retrieval.

In one or more embodiments, connections 116 are links between nodes 112 that define how different medical entities relate to one another. Connections 116 represent relationships. By connecting concepts, e.g., diagnoses, treatments, and symptoms, connections 116 facilitate a comprehensive understanding of complex health data, supporting interoperability, clinical decision support, and efficient data retrieval across various healthcare systems.

In one or more embodiments, relationships 118 define the type and nature of connections between different concepts or nodes 112, e.g., diagnoses, treatments, and symptoms. Common relationships include, “is-a”, e.g., “Type 1 Diabetes is-a Diabetes”, “treats”, e.g., “Insulin treats Diabetes”), and “part-of”, e.g., “Heart is part-of Cardiovascular System”. Relationships 118 organize medical data into meaningful structures, enabling systems to interpret complex medical knowledge, support decision-making, and allow data interoperability across healthcare platforms.

In one or more embodiments, vector embeddings 120 are text that have been converted to a numeric format. The vector embeddings 120 are representations of individual words for text analysis, typically in the form of a real-valued vector. The vector embeddings 120 may represent individual text or may represent an aggregation of text. The vector embeddings 120 may be formed using various word embedding techniques. Vector embeddings 120 represent terms using sets of relationships and/or sets of terms associated with the particular term.

In one or more embodiments, the similarity values or measures 122 in the data repository 102 provide an indication of the similarity between the vector embeddings 120 of a term representing a standard code and a term representing a proprietary code. The higher the similarity measures 122, e.g., the closer to 1.0, the greater a semantic match between the vector embeddings 120. Similarity measures 122 may be weighted to reflect the relevance of the type of data used to calculate the vector embeddings. For example, data with a high relevance to determining an appropriate mapping of a proprietary code may receive a higher weight than data with less relevance to the mapping.

In one or more embodiments, similarity measures 122 that meet or exceed a threshold 124 are identified as semantic matches 126. Threshold 124 is a predefined limit or value that indicates, with reasonable certainty, that two terms are semantic matches 126.

In one or more embodiments, semantic matches 126 are two terms, one from a proprietary code and the other from a standard code, with vector embeddings having a similarity score that exceed threshold 124. Semantic matches 126 are a pair of terms that are determined to represent the same entity or concept. The greater a similarity score between the vector embeddings of the terms, the greater the certainty that the terms are semantic equivalents.

In embodiments, mappings 128 include mappings between proprietary codes 132 and standard codes 130. Mappings 128 may also include mappings between and within standard codes 130 and between and within proprietary codes 132.

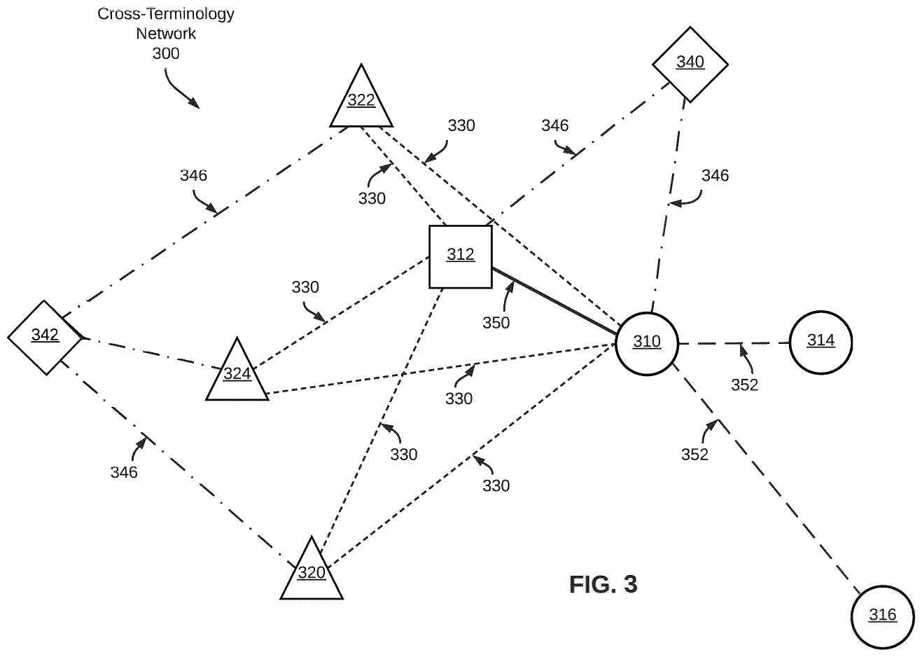

In one or more embodiments, cross-terminology mapping engine 104 refers to hardware and/or software configured to perform operations described herein for mapping proprietary codes with standard codes using similarity between network relationships. Examples of operations for mapping proprietary codes with standard codes using similarity between network relationships are described below with reference to FIG. 3.

In an embodiment, cross-terminology mapping engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, cross-terminology mapping engine 104 includes an ontology editor 134. Ontology editor 134 is software and/or hardware configured to perform operations described for creating, modifying, and managing ontologies, i.e., formal representations of knowledge in a specific domain. Ontology editors enable users to define concepts, specify relationships between the concepts, and organize information hierarchically. Ontology editor 134 is used to build and maintain knowledge models, e.g., cross-terminology network 110, supporting tasks like data integration, semantic search, and decision-making.

In one or more embodiments, ontology editor 134 includes one or more of Protégé, TopBraid Composer, OntoStudio, WebProtege, NeOn Toolkit, and/or Visual Notation for OWL Ontologies (VOWL). Protégé is an open-source ontology editor that supports the creation, visualization, and management of ontologies. Protégé provides tools for defining classes, properties, and relationships, and it supports the OWL (Web Ontology Language) standard. TopBraid Composer is a comprehensive tool for developing, testing, and managing semantic models, including ontologies. TopBraid Compser supports various standards, like OWL, Resource Description Framework (RDF), and SPARQL, and provides a visual interface to build and edit ontologies. OntoStudio is an ontology editor for developing OWL and RDF ontologies. OntoStudio supports advanced features, like reasoning and ontology validation, and includes tools for graphical modeling and Application Programming Interface (API) integration. WebProtege is a web-based version of Protégé that allows users to collaboratively create, edit, and share ontologies online. NeOn Toolkit is an open-source, extensible toolkit for building ontologies and semantic web applications. NeOn Toolkit offers support for multi-ontology development and reasoning. VOWL is visual editor designed to create and understand OWL ontologies.

In one or more embodiments, the cross-terminology mapping engine 104 includes a mapping module 136. The mapping module 136 is software and/or hardware configured to perform the operations described for translating and aligning data between different formats, schemas, or terminologies. Mapping module 136 connects disparate healthcare terminologies (e.g., SNOMED CT, ICD-10, LOINC, and RxNorm) that allow systems to communicate and share data accurately. Mapping module 136 may use tools (e.g., UMLS Metathesaurus, BioPortal, or OMOP) that have pre-existing mappings between common healthcare terminologies. By establishing equivalences and relationships between terms across the terminologies, mapping module 136 supports interoperability, ensuring that clinical data remains consistent and usable across various healthcare platforms and applications.

In one or more embodiments, cross-terminology mapping engine 104 includes a reasoning engine 138. Reasoning engine 138 includes software and/or hardware configured to perform the operation described herein for applying logical rules and algorithms to terminologies to infer new information, validate data relationships, and/or make decisions based on predefined knowledge.

In one or more embodiments, reasoning engine 138 includes one or more of the following: Hermit, FaCT++, Pellet, RacerPro, Snorocket, ELK, and PROTON. Hermit is an OWL reasoner that classifies and checks the consistency of ontologies. FaCT++ is a description logic reasoner optimized for handling complex ontologies, especially those in OWL DL. Pellet is a well-known, open-source OWL DL reasoner that supports SPARQL queries, rule-based reasoning, and consistency checking. RacerPro is a high-performance reasoner for OWL and RDF that supports both standard and complex reasoning tasks. Snorocket is a scalable ontology classifier that efficiently processes large medical terminologies, such as SNOMED CT, and is commonly used in healthcare due to its focus on speed and scalability. ELK is a highly efficient reasoner for OWL 2 EL. PROTON is a rule-based reasoning engine designed to work with ontologies and semantic models that enables advanced query capabilities.

In one or more embodiments, cross-terminology mapping engine 104 includes a vector generator 140. Vector generator 140 includes software and/or hardware for performing one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by machine learning algorithms.

In some embodiments, the vector embedding functions are word embedding techniques. Word embedding techniques use natural language processing (NLP) and machine learning to represent words as dense vectors of real numbers. Word embedding techniques aim to capture the semantic and syntactic meaning of words as well as their relationships with other words in a language.

In one or more embodiments, word embedding techniques include Term Frequency-Inverse Document Frequency (TF-IDF), Word2Vec, Global Vectors (GLOVE), Large Language Models (LLM), and/or BioWordVec fastText. The TF-IDF model is designed to give more weight to the words that are very specific to certain documents but give less weight to the words that are more general and occur across most documents. The Word2Vec model represents words in the form of dense vectors by capturing syntactic (grammar) and semantic (meaning) relationships. Given a large enough dataset, the Word2vec model provides strong estimates about a word's meaning based on its frequency of occurrence in the text. The GLOVE model is an unsupervised learning model that can be used to obtain dense word vectors like the Word2Vec model. The GLOVE model first creates a large word-context, co-occurrence matrix including pairs (word, context). Each element in this matrix represents how often a word or a sequence of words occurs within the context and then applies matrix factorization to approximate this matrix. The BioWordVec fastText model is 200-dimensional word embeddings trained on PubMed and MIMIC-III data and is the extension of the original BioWordVec that provides fastText word embeddings trained using PubMed and MeSH. A subword embedding model used by the BioWordVec fastText model better handles Out of Vocabulary (OOV) tokens and improves the quality of the word embeddings.

In one or more embodiments, the word embedding techniques include Self-Alignment Pretraining for Biomedical Entity Representations (SAPBERT). The SAPBERT model leverages the Unified Medical Language System (UMLS), a comprehensive resource in the biomedical field. UMLS incorporates a vast collection of biomedical concepts and synonyms from various controlled vocabularies, like MeSH, SNOMEDCT, RxNorm, Gene Ontology, and OMIM. This rich source of data greatly enhances the model's understanding of medical terminology and relationships. SAPBERT provides contextual embeddings, meaning that it can understand the meaning of words and phrases in context. This is crucial for understanding complex medical texts and making accurate predictions in healthcare applications. The SAPBERT model can accurately capture fine-grained semantic relationships and heterogeneous naming in the biomedical domain compared to other variants of Bidirectional Encoder Representations from Transformers (BERT). The ability of SAPBERT to handle out-of-vocabulary (OOV) terms, misspelled words, and rare medical terms provides a significant advantage over other models.

In one or more embodiments, cross-terminology mapping engine 104 includes a similarity score calculator 142. Similarity score calculator 142 calculates a similarity between vector embeddings for terms associated with standard codes and vector embeddings for terms associated with proprietary codes. The similarity score calculator 142 may include Facebook AI Similarity Search (FAISS). FAISS is an open-source library developed by Facebook for efficient similarity search and clustering of high-dimensional vectors. FAISS is optimized for both CPU and GPU architectures, enabling fast and scalable similarity search operations on large datasets. FAISS supports a range of similarity metrics, including Euclidean distance, cosine similarity, inner product, and L2 distance. FAISS offers various indexing methods, including the inverted file, Hierarchical Navigable Small World (HNSW), and product quantization. HNSW is an algorithm for efficient similarity searches in high-dimensional spaces. These indexing techniques help speed up nearest-neighbor searches in high-dimensional spaces. In an embodiment, FAISS is combined with HNSW as the indexing approach. FAISS can be integrated with popular machine learning libraries and frameworks, such as PyTorch and TensorFlow, making it easier to incorporate similarity searches into machine learning pipelines. This may lead to significant improvements in the speed and scalability of the similarity search operations. As an open-source library, FAISS is available for developers and researchers to use, modify, and contribute to its development.

In one or more embodiments, cross-terminology mapping engine 104 includes a display module 144. Display module 144 is software and/or hardware configured to perform operations described herein for displaying information to a user. Display module 144 may provide a user-friendly interface that allows a user to interact with the cross-terminology mapping engine 104, confirm semantic matches, and navigate through terminologies.

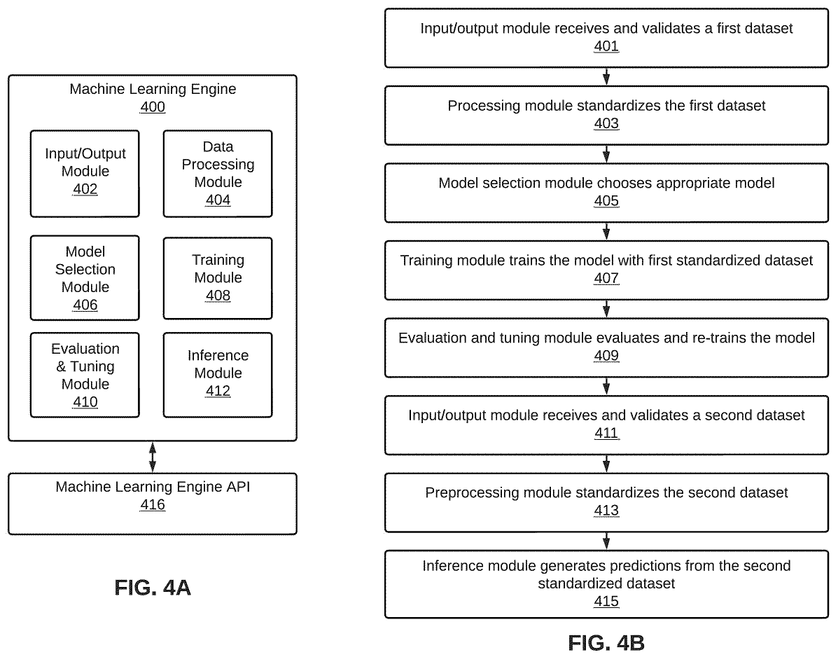

In one or more embodiments, cross-terminology mapping engine 104 includes a machine learning engine 150. Machine learning engine 150 will be described below with reference to FIGS. 4A and 4B.

In one or more embodiments, interface 106 refers to hardware and/or software configured to facilitate communications between a user and cross-terminology mapping engine 104. Interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of interface 106 are specified in different languages. The behavior of user interface elements is specified in a dynamic programming language such as JavaScript. The content of user interface elements is specified in a markup language, such as hypertext markup language (HTML) or XML User Interface Language (XUL). The layout of user interface elements is specified in a style sheet language such as Cascading Style Sheets (CSS). Alternatively, interface 106 is specified in one or more other languages, such as Java, C, or C++.

### 3. Mapping Proprietary Codes with Standard Codes Using Similarity Between Network Relationships

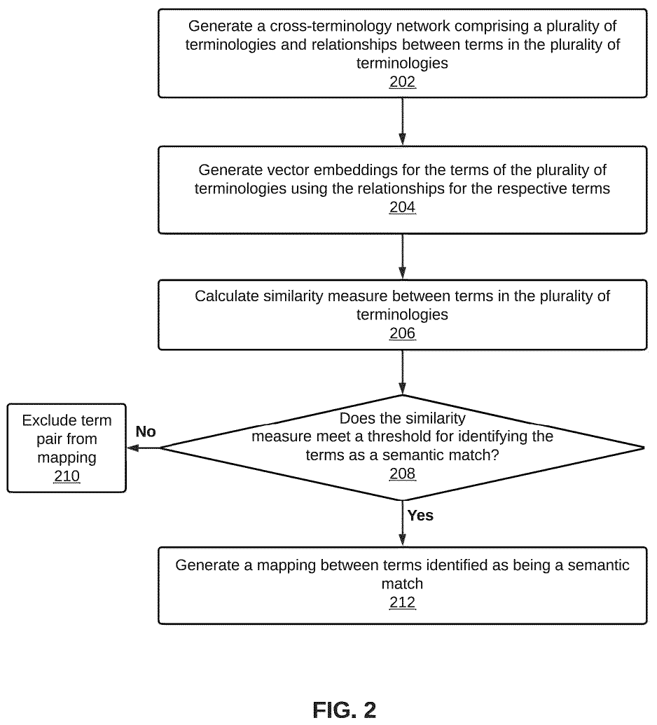

FIG. 2 illustrates an example set of operations for mapping proprietary codes with standard codes using similarity between network relationships in accordance with one or more embodiments. One or more operations illustrated in FIG. 2 may be modified, rearranged, or omitted. Accordingly, the particular sequence of operations illustrated in FIG. 2 should not be construed as limiting the scope of one or more embodiments.

One or more embodiments generate a cross-terminology network comprising a plurality of terminologies and relationships between terms in the plurality of terminologies (Operation 202). Initially, the system receives user input indicating a selection of terminologies to include within the network. The selected terminologies may comprise one or more standard coding systems (e.g., SNOMED CT, LOINC, ICD, CPT, RxNorm) and one or more proprietary or organization-specific code systems (e.g., Multum, Medi-Span, Code Set 72). The number and diversity of terminologies included directly influence the dimensionality and connectivity of the resulting cross-terminology network. A greater number of terminologies yields a richer set of inter-terminology and intra-terminology relationships, thereby enhancing the graph's representational fidelity and enabling more robust similarity computations. The plurality of terminologies may be selected based on the target application domain (e.g., diagnostics, laboratory testing, medications, procedures) or according to a subset of semantic categories of interest. The system may automatically recommend additional terminologies for inclusion by analyzing metadata tags, domain ontologies, or prior mappings within the system's repository.

In one or more embodiments, the system accesses the selected terminologies from one or more data sources. The system may retrieve terminology data through standardized APIs, publicly available repositories, or subscription-based terminological web services. Many standard terminologies, such as SNOMED CT and LOINC, publish formal ontologies encoded in structured data formats, including Comma-Separated Values (CSV), JavaScript Object Notation (JSON), OWL, and RDF. The system may include a data ingestion pipeline configured to parse, normalize, and harmonize these heterogeneous data formats into a common internal representation. During import, the system may extract hierarchical relationships, concept definitions, synonyms, identifiers, and/or version metadata. The system may then store the preprocessed data in a repository made accessible to an ontology editor component. The ontology editor component may begin constructing the cross-terminology network graph. The ontology editor may execute locally within a client application or remotely within a distributed environment connected to a semantic knowledge graph service.

In one or more embodiments, the system uses the ontology editor to construct, modify, and/or visualize the cross-terminology network. The ontology editor represents the network as a set of interconnected ontological classes, object properties, and data properties, enabling both human-readable and machine-interpretable access to terminology relationships. The ontology editor may define explicit cross-terminology object properties, such as equivalentTo, broaderThan, narrowerThan, sameAs, partOf, and relatedTo, to encode various semantic relationships between terms originating from distinct terminologies. The ontology editor may automatically align terms using lexical, syntactic, and/or semantic similarity algorithms, including string matching, synonym expansion, and/or embedding-based similarity detection. The system may leverage mapping tools that include pre-existing relationships between common terminologies (e.g., UMLS Metathesaurus or BioPortal mappings) to bootstrap the creation of inter-terminology links. The system may render visual representations of the network as interactive graphs, allowing users to inspect node-level metadata, navigate hierarchical paths, and/or refine mappings. The ontology editor ensures that the resulting network topology accurately reflects both intra-terminology hierarchies and cross-terminology linkages, establishing a unified semantic framework for subsequent similarity computation and embedding generation.

In one or more embodiments, the system integrates a reasoning engine with the ontology editor to ensure logical consistency and infer additional relationships within the cross-terminology network. The reasoning engine may apply formal logic, description logic (DL), and/or rule-based inference over the ontology's axioms and relationships to validate that the network satisfies defined semantic constraints. The reasoning engine may identify redundant or contradictory relationships, detect orphaned nodes lacking valid links, and/or resolve cycles within the ontology graph. The reasoning engine may execute classification and realization tasks to determine subclass hierarchies and instance memberships dynamically. Based on a predefined set of ontological rules or inference templates, the reasoning engine can infer indirect or transitive connections between terms. For example, the reasoning engine can deduce that if Code A treats Condition X and Condition X is-a Disease Y, then Code A treats Disease Y. Such inferred relationships are automatically added to the cross-terminology network, thereby expanding its connectivity and enriching the semantic context used for downstream vector embedding generation and similarity-based mapping.

One or more embodiments generate vector embeddings for the terms of the plurality of terminologies using the relationships for the respective terms (Operation 204). The system may generate vector embeddings for the terms contained within the plurality of terminologies by encoding each term and its associated relationships into a multi-dimensional numerical representation. Each vector embedding serves as a machine-interpretable representation of the contextual and semantic meaning of the corresponding term. The embedding process begins by aggregating textual and relational information for each term. This aggregated information may include the preferred term label, synonyms, hierarchical relationships (e.g., is-a, part-of, broaderThan, narrowerThan), definitional descriptions, and/or the neighboring node labels in the cross-terminology network. The system may generate a relationship corpus by concatenating these relationship descriptors and term labels into a structured textual sequence. This corpus captures lexical features and structural features derived from the connectivity of each term within the cross-terminology graph.

In one or more embodiments, the system performs pre-processing on the relationship corpus to optimize the relationship corpus for embedding model input. Pre-processing may include converting text to lowercase, normalizing Unicode characters, removing stop words and punctuation, retaining domain-specific numeric tokens (e.g., dosage values or laboratory units), substituting acronyms with standardized forms, and/or applying stemming or lemmatization to unify morphological variants. The system may employ custom pre-processing pipelines tuned for healthcare and biomedical text, incorporating vocabularies from Unified Medical Language System (UMLS) or RxNorm, to retain domain-relevant tokens. Tokenization may be performed using sub word segmentation algorithms, such as byte pair encoding (BPE) or WordPiece, to preserve rare or compound medical terms.

In one or more embodiments, after pre-processing, the system applies a vector embedding function to the textual corpus of each term to produce dense, continuous vector representations. The vector embedding function may correspond to a static or contextual embedding model depending on the configuration. The system may use static models, such as Word2Vec, GloVe, or BioWordVec, to capture co-occurrence patterns from corpus statistics. The system may employ contextual embedding models, such as BioBERT, SapBERT, or ClinicalBERT, to encode contextual semantics using transformer-based self-attention mechanisms. The system may fine-tune the selected embedding model on the terminology corpus to adapt the model's parameters to the specific linguistic and relational patterns of proprietary and standard code systems.

In one or more embodiments, the resulting term embeddings are stored as high-dimensional numerical vectors (e.g., 200-1,024 dimensions) in a vector store or embedding index. Each embedding may capture lexical similarity and relational proximity, so terms with overlapping clinical or functional meaning are located near one another in the embedding space. For example, embeddings for “Hemoglobin Test (LOINC)” and “HGB Observation (Cerner Code Set 72)” may occupy neighboring regions in the vector space due to shared contextual features. Thus, the embeddings encode semantic continuity between terminologies, enabling the system to subsequently compute similarity measures across proprietary codes sets and standard code sets for mapping and interoperability purposes.

In one or more embodiments, additional structural embeddings are computed directly from the topology of the cross-terminology graph using graph embedding algorithms, such as Node2Vec, TransE, or GraphSAGE. These algorithms consider both local connectivity (e.g., direct parent-child relationships) and global graph context (e.g., neighborhood similarity), allowing the system to capture semantic relationships even in cases where textual descriptors differ substantially. The system may concatenate or combine the output of the text-based and graph-based embedding models through weighted averaging to generate hybrid embeddings that jointly represent lexical semantics and/or ontological structure.

One or more embodiments calculate a similarity measure between terms in the plurality of terminologies (Operation 206). A similarity calculation module operates as a mathematical and computational component configured to quantify the degree of semantic equivalence between a term in a proprietary code set and a term in a standard code set. The similarity measure serves as a numerical indicator of how closely two vector embeddings align within a high-dimensional semantic space derived from the embedding models. A higher similarity value indicates a greater degree of semantic correspondence between the two terms, implying that they represent conceptually equivalent or related clinical or operational entities. The system translates the abstract semantic relationships captured in the embedding space into concrete numerical indicators of equivalence, enabling scalable, automated mapping between proprietary and standardized terminologies.

In one or more embodiments, the similarity calculation module receives as input the vector embeddings generated for all terms within the selected terminologies. Each embedding is treated as a vector in an n-dimensional Euclidean space, where n corresponds to the dimensionality of the embedding model (e.g., 200, 512, or 1,024 dimensions). The module performs a pairwise comparison between the vector embeddings of proprietary code terms and those of standard code terms. For scalability, the system may organize embeddings within a vector index optimized for approximate nearest-neighbor (ANN) search. Implementations may utilize different frameworks, such as FAISS or Annoy, to perform large-scale similarity comparisons across millions of vector embeddings efficiently. FAISS supports both CPU and GPU acceleration, allowing similarity computation to execute in parallel over distributed hardware environments.

In one embodiment, the similarity between two embeddings is computed using a cosine similarity metric. Cosine similarity measures the angular closeness between two vectors, irrespective of their magnitudes, providing a normalized metric in the range of −1 to 1. A similarity value approaching 1 indicates a high likelihood that the terms represented by the respective embeddings share equivalent or contextually related meanings. The system may compute similarity using Euclidean distance, inner product, or L2 distance, depending on the training characteristics of the embedding model and the normalization applied during preprocessing. For distance-based metrics, lower values signify greater similarity, and the system may invert or normalize these scores for consistency.

In one or more embodiments, the similarity calculation module applies vector normalization and dimensional weighting prior to similarity computation. Vector normalization ensures that embeddings are scaled to unit length, improving comparability across embeddings generated from heterogeneous data sources. Dimensional weighting assigns higher influence to embedding dimensions that have demonstrated greater discriminative power in previous mapping tasks. Such weights may be learned empirically either by using historical mappings or derived from feature importance metrics computed by the embedding model.

In some embodiments, the similarity calculation is further enhanced through contextual weighting or domain-aware feature fusion. For example, if the embedding vectors include concatenated subcomponents (e.g., text-based embeddings combined with graph-based embeddings), the similarity module may compute a weighted sum of partial similarities across subspaces. This allows the system to account for both linguistic and structural correspondence between codes, improving robustness in scenarios where textual similarity alone may be insufficient.

In one or more embodiments, the similarity module performs index pruning and ANN clustering to reduce computational overhead in large terminology datasets. By grouping vector embeddings into semantically dense clusters, the system limits exhaustive comparisons to subsets of candidates with high likelihood of equivalence. The system may implement hierarchical indexing structures, such as HNSW graphs or inverted file indexes (IVF), to further accelerate retrieval operations. These structures allow sublinear search complexity, making the system suitable for high-scale deployments across thousands of proprietary and standard code systems.

In one or more embodiments, after computing similarity values for relevant term pairs, the system generates a similarity matrix where each entry corresponds to the similarity score between a proprietary term and a standard term. The system may persist the resulting matrix in a vector database or memory store.

One or more embodiments determine that the similarity measure meets a threshold for identifying the terms as semantic match (Operation 208). The threshold serves as a quantitative decision boundary that distinguishes semantically equivalent or closely related terms from dissimilar or unrelated ones. By comparing each similarity measure against the threshold, the system automatically identifies term pairs that represent strong semantic alignment and can be mapped confidently across coding systems.

In one or more embodiments, the threshold is a static value predefined by system configuration (for example, a cosine similarity ≥0.85 or Euclidean distance ≤0.20) or a dynamic threshold that adapts based on data characteristics, domain context, or model performance feedback. Dynamic thresholds may be computed through statistical calibration techniques, such as percentile-based cutoffs, z-score normalization, or clustering of similarity distributions. For instance, if the similarity scores for compared term pairs form a bimodal distribution, the system may identify an optimal cut-point between the modes corresponding to semantically “matched” and “unmatched” clusters using different algorithms, such as Otsu's method or Gaussian Mixture Model (GMM) fitting.

In one or more embodiments, the system performs multi-tiered threshold evaluation that incorporates contextual, domain-specific, and relational confidence signals. For example, the system may apply separate threshold tiers for (a) direct lexical matches, (b) hierarchical relationship matches, and (c) inferred graph-based matches. Each tier may have a distinct threshold tuned to its data type, e.g., 0.95 for identical lexical synonyms, 0.80 for hierarchical relationships, and 0.70 for inferred relationships. The system may dynamically adjust thresholds based on the underlying terminology. For example, mappings between well-structured vocabularies, such as SNOMED CT and LOINC, may require stricter thresholds, whereas mappings between less formal proprietary terminologies may allow lower thresholds to accommodate linguistic variability.

In one or more embodiments, the system evaluates the similarity measure in conjunction with auxiliary confidence parameters or metadata features. For instance, the system may assign higher confidence to mappings where both terms share equivalent hierarchical ancestors or belong to the same semantic type (e.g., both represent laboratory tests or medications). Similarly, the system may integrate contextual weighting from the embedding generation phase, e.g., emphasizing relational embeddings over textual embeddings when the latter have sparse or ambiguous labels. The system may compute a composite confidence score as a weighted function of similarity value, relationship density, ontology depth, and/or metadata consistency. Only pairs with composite scores that exceed a configurable confidence threshold are designated as semantic matches.

In one or more embodiments, the system utilizes a thresholding engine or semantic decision module responsible for evaluating and flagging candidate mappings. The thresholding engine retrieves the similarity values and applies the appropriate decision rule. When the similarity measure for a term pair meets or exceeds the threshold, the pair is flagged as a semantic match, and an association record is generated that links the proprietary code to the corresponding standard code. These association records are written to a mapping repository or persisted as edge relationships in the cross-terminology network. The mapping repository may include versioning metadata, confidence levels, and/or the originating model or embedding version used to generate the mapping.

In one or more embodiments, to improve accuracy and reduce false positives, the system may apply post-threshold validation using auxiliary reasoning and domain constraints. For instance, if two candidate matches exceed the similarity threshold but belong to different semantic categories (e.g., a medication vs. a laboratory test), the reasoning engine may invalidate or deprioritize the mapping. Conversely, when multiple proprietary terms map to a single standard term above threshold, the system may use contextual hierarchy or frequency analysis to retain the most probable match.

In one or more embodiments, the system supports human-in-the-loop verification for mappings near the threshold boundary (e.g., similarity values within a tolerance interval such as ±0.03). In such cases, the display module may present a ranked list of potential matches with confidence indicators, allowing domain experts to confirm, reject, or adjust mappings. The system may log and use expert feedback to retrain or recalibrate thresholds in subsequent iterations, forming an adaptive feedback loop that continuously refines mapping accuracy.

In one or more embodiments, the system maintains threshold metadata across versions and domains to ensure auditability and explainability. Each threshold determination may include various contextual parameters, such as the embedding model version, similarity metric used, vector dimensionality, normalization technique, and/or reasoning constraints applied during evaluation. This metadata allows reproducibility of mapping decisions and supports regulatory or compliance audits, particularly in healthcare environments subject to data provenance requirements.

When the similarity measure for pairs of terms fall below a threshold measure, one or more embodiments exclude the term pairs from mapping (Operation 210). An exclusion operation ensures that only term pairs exhibiting sufficient semantic proximity are persisted as valid mappings between proprietary and standard codes, thereby preserving mapping accuracy and preventing propagation of erroneous relationships across downstream systems. The exclusion of term pairs with similarity measures falling below the threshold serves as a quality-control gate in the mapping pipeline. Exclusion of term pairs enforces semantic integrity, preserves ontological consistency, and/or prevents downstream propagation of inaccurate or weakly correlated mappings, thereby ensuring that only statistically and semantically validated associations contribute to the standardized terminology alignment maintained within the cross-terminology network.

In one or more embodiments, the exclusion process is managed by a filtering module or mapping validation engine integrated within the cross-terminology mapping engine. Exclusion decisions may not simply be binary. The system may assign graded classification labels to each pair, e.g., Accepted, Rejected, or Candidate for Review. The system may immediately filter pairs designated as Rejected from subsequent processing. The system may route pairs within a marginal tolerance range (for example, within 0.02 of the threshold) to a review queue for secondary evaluation or human-in-the-loop adjudication. This multi-tier exclusion approach allows the system to maintain high precision while retaining the flexibility to capture borderline cases that might later be validated through reasoning or expert feedback.

In one or more embodiments, the filtering module implements adaptive exclusion policies that vary by terminology type or embedding model. For example, mappings involving fine-grained medical procedure codes may require higher thresholds than mappings between broader clinical concept hierarchies. The system may adjust exclusion thresholds dynamically, based on domain context, prior false-positive rates, or similarity score distribution patterns observed during runtime. The system may log these adaptive adjustments as part of mapping metadata to maintain traceability.

In one or more embodiments, prior to final exclusion, the system may perform a secondary validation check using contextual or ontological constraints. For instance, if a term pair has a low embedding similarity score but belongs to the same higher-order concept (e.g., both are child terms under “antibiotic medication” in SNOMED CT), the reasoning engine may override the exclusion and flag the pair for re-evaluation. Conversely, if a pair shares superficial lexical similarity but originates from incompatible semantic categories (e.g., a laboratory procedure vs. a diagnostic imaging study), the reasoning engine enforces exclusion even if the similarity score is near the threshold.

In one or more embodiments, excluded term pairs are persisted in a rejection repository or audit log that records the rationale for exclusion, including similarity score, applied threshold, model version, and/or any contextual modifiers used during evaluation. Storing exclusion metadata allows traceability, reproducibility, and potential future reprocessing if embedding models or threshold parameters are updated. For example, when a new embedding model version is deployed or the threshold policy is recalibrated, the system may automatically re-evaluate previously excluded pairs against the updated configuration to identify new potential matches.

In one or more embodiments, the exclusion operation contributes to maintaining graph integrity within the cross-terminology network. By preventing low-similarity term pairs from forming edges, the system avoids introducing noise or spurious relationships that could distort vector embedding retraining or reasoning inference. The pruning of below-threshold edges ensures that the resulting network maintains high semantic precision, directly impacting the performance of downstream similarity calculations, ontology reasoning, and mapping analytics.

In one or more embodiments, the exclusion step may also update various performance metrics, such as precision, recall, and F1-score, using historical validation data or expert-verified mappings as ground truth. The system can compute and monitor exclusion rates to maintain target quality thresholds. These metrics can further inform threshold tuning algorithms, allowing the system to balance false positives and false negatives dynamically based on operational objectives (e.g., prioritizing precision over recall in clinical safety-critical contexts).

One or more embodiments generate a mapping between terms identified as being a semantic match (Operation 212). When the computed similarity measure for a pair of terms meets or exceeds the threshold, the system records a semantic association linking the proprietary term to the corresponding standard term. The generated mapping represents a validated equivalence or near-equivalence relationship that can be consumed by external systems to normalize, translate, or integrate heterogeneous datasets. Each mapping thus constitutes a machine-readable correspondence between two code systems that may differ in naming conventions, data models, or hierarchical structures.

In one or more embodiments, mapping generation is performed by a mapping module within the cross-terminology mapping engine. The module receives as input a filtered list of term pairs that have passed the similarity threshold evaluation. For each accepted pair, the module generates a mapping object, encapsulating the following: (a) the identifiers of the proprietary and standard terms, (b) the similarity score and confidence metrics, (c) the type of semantic relationship (e.g., equivalentTo, narrowerThan, broaderThan), (d) the source embedding model and version, (e) the timestamp and user or process identifier, and/or (f) the validation metadata, such as reasoning outcome or reviewer approval status. The system may assign each mapping object a globally unique mapping identifier (GUID or UUID) and persist each mapping object to a mapping repository implemented as a structured database or graph-based knowledge store. The system may back the repository by a graph database (e.g., Neo4j, Amazon Neptune, or Oracle Spatial and Graph) The system may store the mappings as edges connecting proprietary code nodes and standard code nodes within the cross-terminology network. The edge properties may store confidence values, provenance data, and/or mapping categories, allowing efficient traversal, filtering, and analytic queries.

In one or more embodiments, mappings are also represented in standardized interchange formats, such as RDF, JSON-LD, or FHIR ConceptMap resources, enabling interoperability across external systems. By exporting or synchronizing mappings in these standardized schemas, the system allows EHR systems, data warehouses, and/or interoperability frameworks to consume and apply mappings automatically during data integration workflows.

In one or more embodiments, the system maintains version control and lineage tracking for each mapping. Whenever a new mapping is created, updated, or deprecated, the system records a version entry that captures the originating dataset, embedding model parameters, and/or threshold policy used during generation. This ensures that mappings are reproducible and auditable, supporting regulatory and quality-assurance requirements. The system may retain historical mappings to maintain backward compatibility with earlier system releases or external integration partners.

In one or more embodiments, the mapping module performs bidirectional registration of each mapping. The system updates both the proprietary term record and the standard term record with reciprocal references, indicating the association between the proprietary term record and the standard term record. This allows downstream queries or reasoning engines to traverse relationships in either direction, facilitating different use cases, such as, “Find all proprietary codes equivalent to SNOMED concept X,” or “Determine the standard counterpart for local code Y.”

In one or more embodiments, mappings are assigned confidence tiers (e.g., High, Medium, Low) based on similarity values, model-specific uncertainty measures, or expert validation status. These tiers enable downstream systems to apply mappings selectively according to their required confidence level. For example, an automated billing system may use only High-confidence mappings, whereas a research data-harmonization pipeline may include Medium-confidence mappings to maximize coverage.

In one or more embodiments, the system supports incremental updates and continuous learning. As new proprietary codes are introduced or existing standard terminologies are revised, the system can recompute vector embeddings and similarity measures for only the affected terms, regenerating and versioning mappings incrementally. A feedback loop may incorporate user corrections or expert reviews, retraining the embedding models and recalibrating thresholds to improve future mapping accuracy.

In one or more embodiments, generated mappings are also used to enrich the cross-terminology network. Each new mapping creates an additional inter-terminology edge, increasing the graph's connectivity and supporting higher-order reasoning, such as transitive equivalence discovery or multi-hop semantic inference. The system may periodically recompute derived relationships using the reasoning engine to ensure that related terms across domains remain semantically aligned.

In certain embodiments, the mappings serve as a foundation for data interoperability and semantic normalization across healthcare and enterprise ecosystems. For example, a healthcare data integration service may leverage the mappings to translate local laboratory test codes into LOINC identifiers for clinical data exchange, whereas a clinical analytics platform may normalize medication codes from disparate hospital systems into RxNorm identifiers to enable comparative reporting. Similarly, the system may use mappings in real-time API calls or extract-transform-load (ETL) processes to harmonize coded data before ingestion into centralized data warehouses or federated query engines.

In one or more embodiments, the system transforms similarity-based equivalence findings into persistent, verifiable, and/or reusable mapping artifacts. These mappings operationalize the machine-derived semantic relationships discovered in previous stages, providing a reliable mechanism for cross-system interoperability, terminology harmonization, and semantic data integration across heterogeneous proprietary and standardized code environments.

In one or more embodiments, the mappings between terms identified as semantic matches are leveraged to enable a variety of operational, analytical, and/or interoperability functions across healthcare and enterprise data ecosystems. These mappings serve as the foundational layer for semantic normalization, ensuring that data originating from heterogeneous systems, formats, or proprietary vocabularies can be interpreted uniformly within a standardized framework.

In one or more embodiments, the mappings are used to facilitate interoperability between disparate healthcare information systems. For example, when a local hospital information system encodes laboratory tests using a proprietary code set, the mappings allow the results to be translated automatically into corresponding LOINC identifiers for cross-institutional exchange via HL7 FHIR or Clinical Document Architecture (CDA) interfaces. Similarly, the system can convert diagnostic and procedure codes recorded using local terminologies into ICD, SNOMED CT, or CPT codes, enabling consistent interpretation and analytics across EHR systems, payer platforms, and/or clinical registries.

In one or more embodiments, the mappings are used to normalize and integrate data across heterogeneous repositories. During ETL or data pipeline operations, the system applies the stored mappings to reconcile disparate code sets into a unified semantic model before the data is loaded into a data warehouse or federated query engine. This allows analytics, population health reporting, and ML pipelines to operate on harmonized datasets without requiring manual code reconciliation. For example, clinical measures, such as “Hemoglobin A1C” or “Blood Pressure,” may be aggregated across multiple source systems that use different proprietary labels but are normalized to the same LOINC identifiers through the generated mappings.

In one or more embodiments, the mappings are used to augment clinical decision support (CDS) and prior authorization workflows. By mapping local EHR codes to standard terminologies, such as SNOMED CT, RxNorm, or CPT, the system enables rule engines and AI models trained on standardized datasets to operate seamlessly in environments using custom or legacy code sets. This allows automated prior-authorization systems, order validation modules, or drug-drug interaction checkers to reason over standardized representations of proprietary data.

In one or more embodiments, the mappings are utilized to enable regulatory and billing compliance. Standardized mappings ensure that clinical events, diagnoses, and procedures captured using proprietary codes can be accurately transformed into standardized billing codes (e.g., ICD-10-CM or HCPCS) required for payer submissions, claims processing, and quality reporting programs. This reduces administrative burden and ensures conformance with payer and regulatory requirements, such as CMS, Healthcare Effectiveness Data and Information Set (HEDIS), or Office of the National Coordinator for Health Information Technology (ONC) interoperability mandates.

In one or more embodiments, the mappings are used to train, evaluate, and/or calibrate artificial intelligence (AI) models. By providing standardized identifiers for equivalent terms across domains, the system allows training datasets derived from heterogeneous EHR sources to be semantically aligned, ensuring that AI models receive consistent input features regardless of local coding schemes. This enhances model generalizability and facilitates cross-site transfer learning. For example, embeddings generated for mapped terms can be aggregated to construct domain-specific knowledge graphs or improve context-aware clinical reasoning models.

In one or more embodiments, mappings are used for semantic search and knowledge retrieval. When a user or external system issues a search query using a proprietary term, the system expands the query through the mappings to include equivalent standard terms and synonyms. This improves recall and relevance in query results across document repositories, clinical knowledge bases, and federated databases. Similarly, when an analytics platform performs cohort identification, mapped terms ensure inclusion of patients documented under different terminologies but representing the same clinical condition.

In one or more embodiments, the mappings are leveraged to enhance data visualization, reporting, and auditing. Dashboards and analytics reports can display both proprietary and standard term representations, ensuring transparency and interpretability for clinical, operational, and compliance stakeholders. The mappings allow analysts to trace normalized metrics back to their original local codes, supporting data lineage, reproducibility, and regulatory auditability.

In one or more embodiments, the mappings support bidirectional translation and data exchange between systems operating in different terminology domains. For example, a clinical data interface may convert incoming standardized FHIR resources into local proprietary codes for internal processing and then use the same mappings in reverse to translate outbound data back into standard terminologies for external transmission. This bidirectional capability ensures interoperability while preserving local customization within proprietary systems.

In one or more embodiments, the mappings are used to continuously improve and expand the cross-terminology network. As mappings accumulate, the system forms a dense set of validated edges within the ontology graph, allowing reasoning engines to infer indirect equivalences and discover previously unmapped relationships. These expanded relationships can then be re-embedded, improving the performance of future similarity computations and reinforcing the adaptive, self-improving nature of the system.

In one or more embodiments, the mappings between semantically matched terms enable data standardization, clinical reasoning, regulatory compliance, and/or advanced analytics across distributed, heterogeneous, and/or evolving data ecosystems. By transforming local and proprietary code systems into standardized semantic equivalents, the system enables scalable, explainable, and automated interoperability across the full spectrum of healthcare and enterprise data workflows.

In one or more embodiments, after mapping an unmapped or proprietary code to a standardized medical terminology code, the system performs diagnostic determination based on the mapped standard code. The system may carry out the diagnostic determination by a diagnostic inference module operating in conjunction with one or more trained machine learning models and a rules-based inference engine.

In one or more embodiments, the diagnostic inference module receives as input a mapping result that links an unmapped proprietary data element, such as a local code, textual descriptor, or free-form measurement, to a standardized code (for example, a Logical Observation Identifiers Names and Codes (LOINC) code, SNOMED CT concept, or ICD-10-CM diagnosis code). The module then retrieves corresponding reference attributes, such as normal ranges, interpretation rules, and disease associations, from a clinical terminology knowledge base stored in system memory.

In an example, a healthcare laboratory information system records an unmapped data string, “HGB low”, originating from a legacy analyzer interface. The mapping subsystem applies a vector embedding model to the textual token and contextual attributes, generating a vector representation that is semantically compared to known standard code embeddings. The similarity computation identifies LOINC 718-7 (Hemoglobin [Mass/volume] in Blood) as the most semantically aligned standard code. The mapped record now contains the normalized hemoglobin observation and its numeric result (e.g., 8.1 g/dL). The diagnostic inference module interprets the mapped code in the context of clinical parameters such as patient age, sex, and reference intervals. Responsive to detecting that the mapped hemoglobin value is below a clinically defined threshold (e.g., <12.0 g/dL for adult females), the module consults an anemia classification model trained on labeled datasets of laboratory profiles. The model outputs a confidence score (e.g., 0.86) for a target diagnosis—iron-deficiency anemia.

In one or more embodiments, the system transmits the diagnostic determination and its confidence score to a CDS interface integrated with the EHR. The CDS interface presents a structured suggestion such as “Probable iron-deficiency anemia; recommend iron studies and reticulocyte count.” Clinician confirmation of the suggestion writes a provisional diagnosis (SNOMED CT 87522002) into the patient problem list and time-stamps the mapping provenance.

In one or more embodiments, the system includes a treatment initiation module that automatically or semi-automatically initiates a treatment order set based on one or more mapped standard codes. The treatment initiation module operates downstream from the mapping and diagnostic determination modules, leveraging the standardized codes to trigger corresponding order templates, medication protocols, and/or clinical pathways. Upon confirmation of the mapping “HGB low”→LOINC 718-7 and the derived diagnostic determination of anemia, the treatment initiation module may access a clinical rules repository containing protocol definitions indexed by standard codes. Each protocol definition may specify a set of actions, such as ordering follow-up laboratory tests, initiating medication therapy, scheduling procedures, or generating nursing instructions, along with triggering conditions and dependencies.

In an example, responsive to an anemia-related mapping, the module retrieves a “CBC follow-up and iron study” order set, which includes the following orders: (a) Complete blood count (LOINC 57021-8); (b) Ferritin (LOINC 2276-4); (c) Iron and total iron-binding capacity (TIBC) (LOINC 2498-4, 2500-7); (d) Fecal occult blood test (LOINC 57905-2). The module generates an order set object in a structured electronic format (e.g., FHIR Order Bundle) and populates it with patient identifiers, mapped codes, timestamps, and provenance metadata. The order set object is transmitted via an API to the EHR's order-entry subsystem.

In one or more embodiments, the system automatically queues a medication recommendation, such as initiating oral ferrous sulfate therapy, based on the mapped diagnosis and applicable clinical guidelines. The system may present the medication recommendation to the clinician for review and authorization through a secure interface.

### 4. Example Cross-Terminology Network Mapping

A detailed example is described below for purposes of clarity. Components and/or operations described below should be understood as one specific example that may not be applicable to certain embodiments. Accordingly, components and/or operations described below should not be construed as limiting the scope of any of the claims.

FIG. 3 illustrates an example cross-terminology network 300 for mapping a standardized laboratory test code, such as a Hemoglobin Test (LOINC), to a corresponding proprietary or local code such as HGB Observation (Cerner Code Set 72). In the illustrated embodiment, network 300 represents relationships among a plurality of heterogeneous terminology nodes and their associated semantic, attribute, and provenance relationships as determined by one or more vector embedding models and attribute-guarding rules.

Cross-terminology network 300 includes a plurality of nodes representing data concepts, attributes, and provenance elements, and a plurality of edges representing weighted relationships among those nodes. A standard code node 310 corresponds to the Hemoglobin Test (LOINC) and stores metadata identifying the test's official LOINC code, description, and clinical attributes. A proprietary code node 312 corresponds to the HGB Observation within Cerner Code Set 72 and includes metadata for the local code identifier, display label, and mapping attributes.

Cross-terminology network includes additional related and unrelated concept nodes. As shown, first concept node 314 for Hematocrit Test represents semantically related laboratory tests within the same diagnostic domain, and second concept node 316 for Glucose Test represents a lower-similarity concept within the embedding space.

A set of attribute nodes, e.g., first attribute node 320 for Specimen: Blood, second attribute node 322 for Property: Mass/Volume, and third attribute node 324 for Units: g/dL, represent clinical guard conditions used for validating mapping consistency across terminologies. Each attribute node may be connected to both the LOINC node, e.g., standard code node 310, and the Cerner node, e.g., proprietary code node 312, through respective has_attribute edges 330, thereby enforcing that the mapped codes share equivalent specimen type, measurement property, and unit semantics.

Cross-terminology network 300 further includes provenance nodes, i.e., first provenance node 340 and second provenance node 342, identifying the computational or logical origin of the mapping. First provenance node 340 corresponds to a model node labeled Model: ClinicalEmbedding-v1, representing the vector embedding model used to generate code embeddings and compute cosine similarity measures. Second provenance node 342 corresponds to Rule: AttributeGuard-v2, representing the rule-based validation engine applied to enforce attribute equivalence constraints. Provenance nodes are connected to their governed or derived nodes through respective derived_from or governs edges 346.

A mapping edge 350 connects the LOINC Hemoglobin Test node, i.e., standard code node 310, to the Cerner HGB Observation node, i.e., proprietary code node 312. Mapping edge 350 is assigned a mapping weight, e.g., 0.9997, determined by the cosine similarity between the embedding vectors generated for each concept by the embedding model. Mapping edge 350, visually represented as a solid line in FIG. 3, signifies a confirmed equivalence mapping.

Similarity edges 352 connect the LOINC Hemoglobin Test node, i.e., standard code node 310, to other concept nodes within the embedding space. As illustrated, Hematocrit Test node, i.e., first concept node 314, exhibits a moderate similarity value (≈0.9978), reflecting semantic proximity within the same hematology domain, whereas Glucose Test node, i.e., second concept node 316, exhibits a lower similarity (≈0.32), reflecting semantic dissimilarity.

Attribute edges 330 connect the code nodes, i.e., standard code node 310 and proprietary code node 312, to their attribute nodes, i.e., first attribute node 320, second attribute node 322, third attribute node 324, ensuring that mapped codes share equivalent or compatible attributes. The system may automatically invalidate or downgrade mappings if any required attribute node is missing or inconsistent between the two terminologies.

Provenance edges 346 connect the model and rule nodes to their corresponding derived or governed entities. This enables full auditability of the mapping process, allowing downstream systems to reconstruct how a mapping decision was generated, the model and rule set versions used, and when the mapping was validated.

The system applies the ClinicalEmbedding-v1 model to textual descriptors and synonyms of the candidate codes to generate embedding vectors in a shared latent space. The system then computes similarity scores between the LOINC Hemoglobin Test embedding and embeddings of candidate proprietary codes. When a similarity score exceeds a configurable threshold (e.g., 0.90) and the attribute guard validation rules confirm equivalence for the specimen, property, and units, the system instantiates a mapping edge 350 between the corresponding nodes in the cross-terminology network 300.

The system may annotate each edge within the cross-terminology network with different metadata fields, including similarity score, model version, rule version, timestamp, and mapping status (e.g., proposed, validated, deprecated). The resulting cross-terminology network 300 thus provides a machine-interpretable graph representation of cross-terminology relationships that combines semantic similarity evidence with structured attribute validation and provenance traceability.

By representing mappings in this graph-based structure, embodiments of the system enable downstream components to perform federated reasoning, conflict detection, and semantic translation across heterogeneous healthcare coding systems. In this manner, cross-terminology network 300 improves the accuracy, auditability, and automation of terminology alignment operations relative to manual mapping techniques.

### 5. Machine Learning Architecture

FIG. 4A illustrates a machine learning engine 400 in accordance with one or more embodiments. As illustrated in FIG. 4A, machine learning engine 400 includes input/output module 402, data preprocessing module 404, model selection module 406, training module 408, evaluation and tuning module 410, and inference module 412.

In accordance with an embodiment, input/output module 402 serves as the primary interface for data entering and exiting the system, managing the flow and integrity of data. This module may accommodate a wide range of data sources and formats to facilitate integration and communication within the machine learning architecture.

In an embodiment, an input handler within input/output module 402 includes a data ingestion framework capable of interfacing with various data sources, such as databases, APIs, file systems, and real-time data streams. This framework is equipped with functionalities to handle different data formats (e.g., CSV, JSON, XML) and efficiently manage large volumes of data. It includes mechanisms for batch and real-time data processing that enable the input/output module 402 to be versatile in different operational contexts, whether processing historical datasets or streaming data.

In accordance with an embodiment, input/output module 402 manages data integrity and quality as it enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the machine learning process.

In an embodiment, an output handler within input/output module 402 includes an output framework designed to handle the distribution and exportation of outputs, predictions, or insights. Using the output framework, input/output module 402 formats these outputs into user-friendly and accessible formats, such as reports, visualizations, or data files compatible with other systems. Input/output module 402 also ensures secure and efficient transmission of these outputs to end-users or other systems in an embodiment and may employ encryption and secure data transfer protocols to maintain data confidentiality.

In accordance with an embodiment, data preprocessing module 404 transforms data into a format suitable for use by other modules in machine learning engine 400. For example, data preprocessing module 404 may transform raw data into a normalized or standardized format suitable for training ML models and for processing new data inputs for inference. In an embodiment, data preprocessing module 404 acts as a bridge between the raw data sources and the analytical capabilities of machine learning engine 400.

In an embodiment, data preprocessing module 404 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods like imputation or removal of incomplete records, depending on the nature and volume of the missing data. Data preprocessing module 404 may be configured to handle anomalies in different ways depending on context. Data preprocessing module 404 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model's ability to learn effectively.

In an embodiment, data preprocessing module 404 includes a feature encoding framework that ensures categorical variables are transformed into a format that can be easily interpreted by machine learning algorithms. Techniques like one-hot encoding or label encoding may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 404 processes new data for inference, data preprocessing module 404 replicates the same preprocessing steps to ensure consistency with the training data format. This helps to avoid discrepancies between the training data format and the inference data format, thereby reducing the likelihood of inaccurate or invalid model predictions.

In an embodiment, model selection module 406 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 406 employs a variety of statistical and analytical techniques to understand data patterns, identify potential correlations, and assess the complexity of the task. Based on this analysis, it then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 406 utilizes techniques from the field of Automated Machine Learning (AutoML). AutoML systems automate the process of model selection by rapidly prototyping and evaluating multiple models. They use techniques like Bayesian optimization, genetic algorithms, or reinforcement learning to explore the model space efficiently. Model selection module 406 may use these techniques to evaluate each candidate model based on performance metrics relevant to the task. For example, accuracy, precision, recall, or F1 score may be used for classification tasks and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The mean squared error (MSE) metric may be used for regression tasks. MSE measures the average squared difference between the actual and predicted values, providing an indication of the model's accuracy. A lower MSE may indicate a model's greater accuracy in predicting values, as it represents a smaller average discrepancy between the actual and predicted values.

In accordance with an embodiment, model selection module 406 also considers computational efficiency and resource constraints. This is meant to help ensure the selected model is both accurate and practical in terms of computational and time requirements. In an embodiment, certain features of model selection module 406 are configurable such as a configured bias toward (or against) computational efficiency.

In accordance with an embodiment, training module 408 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly. Training module 408 handles the iterative process of feeding the training data into the model, adjusting the model's internal parameters (like weights in neural networks) through backpropagation and optimization algorithms, such as stochastic gradient descent or other algorithms providing similarly useful results.

In accordance with an embodiment, training module 408 manages overfitting, where a model learns the training data too well, including its noise and outliers, at the expense of its ability to generalize to new data. Techniques such as regularization, dropout (in neural networks), and early stopping are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 408 includes logic to handle different types of data and learning tasks. For instance, it includes different training routines for supervised learning (where the training data comes with labels) and unsupervised learning (without labeled data). In the case of deep learning models, training module 408 also manages the complexities of training neural networks that include initializing network weights, choosing activation functions, and setting up neural network layers.

In an embodiment, evaluation and tuning module 410 incorporates dynamic feedback mechanisms and facilitates continuous model evolution to help ensure the system's relevance and accuracy as the data landscape changes. Evaluation and tuning module 410 conducts a detailed evaluation of a model's performance. This process involves using statistical methods and a variety of performance metrics to analyze the model's predictions against a validation dataset. The validation dataset, distinct from the training set, is instrumental in assessing the model's predictive accuracy and its capacity to generalize beyond the training data. The module's algorithms meticulously dissect the model's output, uncovering biases, variances, and the overall effectiveness of the model in capturing the underlying patterns of the data.

In an embodiment, evaluation and tuning module 410 performs continuous model tuning by using hyperparameter optimization. Evaluation and tuning module 410 performs an exploration of the hyperparameter space using algorithms, such as grid search, random search, or more sophisticated methods like Bayesian optimization. Evaluation and tuning module 410 uses these algorithms to iteratively adjust and refine the model's hyperparameters—settings that govern the model's learning process but are not directly learned from the data—to enhance the model's performance. This tuning process helps to balance the model's complexity with its ability to generalize and attempts to avoid the pitfalls of underfitting or overfitting.

In an embodiment, evaluation and tuning module 410 integrates data feedback and updates the model. Evaluation and tuning module 410 actively collects feedback from the model's real-world applications, an indicator of the model's performance in practical scenarios. Such feedback can come from various sources depending on the nature of the application. For example, in a user-centric application like a recommendation system, feedback might comprise user interactions, preferences, and responses. In other contexts, such as predicting events, it might involve analyzing the model's prediction errors, misclassifications, or other performance metrics in live environments.

In an embodiment, feedback integration logic within evaluation and tuning module 410 integrates this feedback using a process of assimilating new data patterns, user interactions, and error trends into the system's knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and tuning module 410 employs version control mechanisms to track changes, modifications, and the evolution of the model, facilitating transparency and allowing for rollback if necessary. This continuous learning and adaptation cycle, driven by real-world data and feedback, helps to endure the model's ongoing effectiveness, relevance, and accuracy.

In an embodiment, inference module 412 transforms data raw data into actionable, precise, and contextually relevant predictions. In addition to processing and applying a trained model to new data, inference module 412 may also include post-processing logic that refines the raw outputs of the model into meaningful insights.

In an embodiment, inference module 412 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 412 transforms the outputs of a trained model into definitive classifications. Inference module 412 employs the underlying model as a tool to generate probabilistic outputs for each potential class. It then engages in an interpretative process to convert these probabilities into concrete class labels.

In an embodiment, when inference module 412 receives the probabilistic outputs from the model, it analyzes these probabilities to determine how they are distributed across some or every potential class. If the highest probability is not significantly greater than the others, inference module 412 may determine that there is ambiguity or interpret this as a lack of confidence displayed by the model.

In an embodiment, inference module 412 uses thresholding techniques for applications where making a definitive decision based on the highest probability might not suffice due to the critical nature of the decision. In such cases, inference module 412 assesses if the highest probability surpasses a certain confidence threshold that is predetermined based on the specific requirements of the application. If the probabilities do not meet this threshold, inference module 412 may flag the result as uncertain or defer the decision to a human expert. Inference module 412 dynamically adjusts the decision thresholds based on the sensitivity and specificity requirements of the application, subject to calibration for balancing the trade-offs between false positives and false negatives.

In accordance with an embodiment, inference module 412 contextualizes the probability distribution against the backdrop of the specific application. This involves a comparative analysis, especially in instances where multiple classes have similar probability scores, to deduce the most plausible classification. In an embodiment, inference module 412 may incorporate additional decision-making rules or contextual information to guide this analysis, ensuring that the classification aligns with the practical and contextual nuances of the application.

In regression models, where the outputs are continuous values, inference module 412 may engage in a detailed scaling process in an embodiment. Outputs, often normalized or standardized during training for optimal model performance, are rescaled back to their original range. This rescaling involves recalibration of the output values using the original data's statistical parameters, such as mean and standard deviation, ensuring that the predictions are meaningful and comparable to the real-world scales they represent.

In an embodiment, inference module 412 incorporates domain-specific adjustments into its post-processing routine. This involves tailoring the model's output to align with specific industry knowledge or contextual information. For example, in financial forecasting, inference module 412 may adjust predictions based on current market trends, economic indicators, or recent significant events, ensuring that the outputs are both statistically accurate and practically relevant.

In an embodiment, inference module 412 includes logic to handle uncertainty and ambiguity in the model's predictions. In cases where inference module 412 outputs a measure of uncertainty, such as in Bayesian inference models, inference module 412 interprets these uncertainty measures by converting probabilistic distributions or confidence intervals into a format that can be easily understood and acted upon. This provides users with both a prediction and an insight into the confidence level of that prediction. In an embodiment, inference module 412 includes mechanisms for involving human oversight or integrating the instance into a feedback loop for subsequent analysis and model refinement.

In an embodiment, inference module 412 formats the final predictions for end-user consumption. Predictions are converted into visualizations, user-friendly reports, or interactive interfaces. In some systems, like recommendation engines, inference module 412 also integrates feedback mechanisms, where user responses to the predictions are used to continually refine and improve the model, creating a dynamic, self-improving system.

### 6. Machine Learning Engine Operations

FIG. 4B illustrates the operation of a machine learning engine in one or more embodiments. In an embodiment, input/output module 402 receives a dataset intended for training (Operation 401). This data can originate from diverse sources, like databases or real-time data streams, and in varied formats, such as CSV, JSON, or XML. Input/output module 402 assesses and validates the data, ensuring its integrity by checking for consistency, data ranges, and types.

In an embodiment, training data is passed to data preprocessing module 404. Here, the data undergoes a series of transformations to standardize and clean it, making it suitable for training ML models (Operation 403). This involves normalizing numerical data, encoding categorical variables, and handling missing values through techniques like imputation.

In an embodiment, prepared data from the data preprocessing module 404 is then fed into model selection module 406 (Operation 405). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 408 trains the selected model with the prepared dataset (Operation 407). It implements learning algorithms to adjust the model's internal parameters, optimizing them to identify patterns and relationships in the training data. Training module 408 also addresses the challenge of overfitting by implementing techniques, like regularization and early stopping, ensuring the model's generalizability.

In an embodiment, evaluation and tuning module 410 evaluates the trained model's performance using the validation dataset (Operation 409). Evaluation and tuning module 410 applies various metrics to assess predictive accuracy and generalization capabilities. It then tunes the model by adjusting hyperparameters, and if needed, incorporates feedback from the model's initial deployments, retraining the model with new data patterns identified from the feedback.

In an embodiment, input/output module 402 receives a dataset intended for inference. Input/output module 402 assesses and validates the data (Operation 411).

In an embodiment, data preprocessing module 404 receives the validated dataset intended for inference (Operation 413). Data preprocessing module 404 ensures that the data format used in training is replicated for the new inference data, maintaining consistency and accuracy for the model's predictions.

In an embodiment, inference module 412 processes the new data set intended for inference, using the trained and tuned model (Operation 415). It applies the model to this data, generating raw probabilistic outputs for predictions. Inference module 412 then executes a series of post-processing steps on these outputs, such as converting probabilities to class labels in classification tasks or rescaling values in regression tasks. It contextualizes the outputs as per the application's requirements, handling any uncertainty in predictions and formatting the final outputs for end-user consumption or integration into larger systems.

In an embodiment, machine learning engine API 414 allows for applications to leverage machine learning engine 400. In an embodiment, machine learning engine API 414 may be built on a RESTful architecture and offer stateless interactions over standard HTTP/HTTPS protocols. Machine learning engine API 414 may feature a variety of endpoints, each tailored to a specific function within machine learning engine 400. In an embodiment, endpoints such as /submitData facilitate the submission of new data for processing, while /retrieveResults is designed for fetching the outcomes of data analysis or model predictions. The MLE API may also include endpoints like /updateModel for model modifications and /trainModel to initiate training with new datasets.

In an embodiment, machine learning engine API 414 is equipped to support SOAP-based interactions. This extension involves defining a WSDL (Web Services Description Language) document that outlines the API's operations and the structure of request and response messages. In an embodiment, machine learning engine API 414 supports various data formats and communication styles. In an embodiment, machine learning engine API 414 endpoints may handle requests in JSON format or any other suitable format. For example, machine learning engine API 414 may process XML, and it may also be engineered to handle more compact and efficient data formats, such as Protocol Buffers or Avro, for use in bandwidth-limited scenarios.

In an embodiment, machine learning engine API 414 is designed to integrate WebSocket technology for applications necessitating real-time data processing and immediate feedback. This integration enables a continuous, bi-directional communication channel for a dynamic and interactive data exchange between the application and machine learning engine 400.

### 7. Generative AI Models

A generative AI model is a machine learning model that is capable of generating new data instances based on the data used to train the model. A generative model may be referred to as a “generative AI model.” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a large language model. Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a softmax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 402, when used for large language models, handles textual data, converting input text into a format that the model can process. This typically involves tokenization, where the text is broken down into manageable pieces, such as words or subwords, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 404 in the context of large language models may include steps such as normalization, where the text is converted to a uniform case and punctuation is standardized. This process ensures that the model treats similar words or symbols consistently, reducing the complexity of the input space. Additionally, techniques such as sentence segmentation may be applied to manage longer texts, enabling the model to process information in chunks that align with natural language structures.

In accordance with one or more embodiments, model selection module 406, when used for large language models involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 408, when used for large language models, is configured to adjust the model's parameters through exposure to training data. This process utilizes optimization algorithms, such as stochastic gradient descent, to minimize the difference between the model's predictions and the actual desired outputs. The training process is computationally intensive, often requiring specialized hardware such as GPUs (Graphics Processing Units) or TPUs (Tensor Processing Units) to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model's performance on new data).

In accordance with one or more embodiments, evaluation and tuning module 410 assesses the performance of large language models using metrics such as perplexity, accuracy, and F1 score, depending on the specific language tasks. Evaluation may involve comparing the model's output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 412, in the context of large language models, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

The self-attention mechanism, which is part of a transformer network, enables the model to weigh the importance of different elements within an input sequence, regardless of their position. This allows the model to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### 8. Practical Applications, Advantages, and Improvements

Embodiments provide several practical applications, advantages, and improvements over existing manual and rule-based terminology mapping approaches. These advantages include automated semantic mapping using ML techniques, improved processing performance for large-scale cross-terminology datasets, enhanced accuracy in code equivalence identification, and increased interoperability between heterogeneous healthcare and enterprise information systems.

Existing systems typically depend on static crosswalk tables, manual curation, and/or lexical string-matching rules that require significant human intervention and fail to adapt to evolving data standards. Such systems are not optimized for high-dimensional similarity computation and often produce inconsistent mappings across domains. Embodiments described herein apply AI and vector embedding models executed by hardware processors to dynamically learn, compute, and maintain mappings. These operations improve the functioning of a computer system by transforming unstructured or heterogeneous code data into standardized, machine-interpretable relationships that can be processed automatically by downstream computing components.

Embodiments improve mapping precision by generating high-dimensional vector embeddings that capture semantic, syntactic, and relational context for each term. These embeddings enable similarity comparison beyond mere text matching. For example, the system can accurately identify “HGB Observation (Cerner Code Set 72)” and “Hemoglobin [LOINC: 718-7]” as semantic equivalents despite differences in terminology or spelling. This results in a measurable reduction in false-positive mappings compared to conventional dictionary-based techniques.

Embodiments perform large-scale similarity computations using optimized vector-indexing algorithms, such as FAISS or Hierarchical Navigable Small World (HNSW) structures. These algorithms allow the mapping engine to execute millions of comparisons in parallel across distributed CPU or GPU resources. As a result, the system significantly reduces processing time for ontology alignment (e.g., from hours or days using manual or rule-based methods to near-real-time computation) while maintaining accuracy across terabyte-scale terminology datasets. This increased throughput provides both a technical benefit (improved computational performance) and an economic benefit (reduced infrastructure and labor costs).

Embodiments ensure that only mappings with similarity measures exceeding an adaptive confidence threshold are recorded, preventing spurious associations and ensuring semantic integrity across connected systems. For instance, proprietary medication codes can be automatically aligned to RxNorm identifiers, allowing EHR systems, payer platforms, and pharmacy databases to communicate consistently. This technical improvement eliminates redundant manual integration steps and improves the reliability of clinical and operational data exchange.

Embodiments improve upon existing approaches by reducing latency, error propagation, and computational redundancy through parallelized embedding generation, efficient indexing, and automated thresholding. These improvements optimize how hardware processors allocate resources, reducing overall compute cycles required per mapping operation.

Embodiments improve the performance of software executing on the computing device by reducing processing overhead associated with rule-based logic and lookup table traversal. The AI-driven similarity computation pipeline processes high-dimensional data directly in vector space, enabling the system to generate and update mappings asynchronously without blocking other workflows.

Embodiments provide a technical benefit, by enabling self-updating, AI-driven mapping models that learn from feedback, and an economical benefit, by reducing the need for continuous human curation. The resulting lower maintenance cost and improved automation efficiency provide a quantifiable return on computational investment for large healthcare enterprises managing millions of codes across multiple systems.

Embodiments implementing semantic interoperability across heterogeneous systems enable a broad range of real-world applications. At a high level, the system may cause automated normalization of data transmitted between EHR systems, analytics platforms, or claims databases. More specifically, the system may cause the following: (a) generation of standardized FHIR resources for outbound data exchange, (b) a CDS engine to trigger rule-based alerts using normalized standard codes, (c) predictive AI models to process harmonized data inputs for patient risk scoring or prior authorization, and/or (d) analytics pipelines to generate unified performance metrics across multi-hospital networks. Each practical application is implemented programmatically by hardware processors executing the machine-learned mapping instructions, rather than abstract human judgment.

One or more embodiments improve the functioning of a computer system by converting unstructured, heterogeneous coded data into structured, machine-readable mappings that can be efficiently queried, reasoned over, and transmitted. This transformation provides a technical benefit—faster, more accurate data processing across distributed computing environments—and an economic benefit by reducing the cost and time associated with maintaining and reconciling multiple code systems. The practical application distinguishes existing approaches by enabling automated, model-driven semantic interoperability rather than relying on static, rule-based crosswalks or manual review.

In an embodiment, the system improves adaptive learning and version control for mapping repositories. The improvement is achieved through continuous feedback loops in which expert corrections or new terminology releases automatically retrain the embedding models and recalibrate similarity thresholds. For example, when a new proprietary laboratory code is introduced, the system autonomously determines its closest standard equivalent, adds the mapping to the repository, and version-controls the change for auditability and reproducibility. This adaptive improvement ensures that the system evolves dynamically with domain changes without re-engineering the underlying software.

By providing automated, AI-driven, and versioned semantic mappings, embodiments address a concrete technical problem—heterogeneous and incompatible coding schemas—that has long impeded interoperability and data exchange in healthcare and enterprise information systems. The solution improves computer system performance, reduces manual labor, and increases confidence in downstream analytics and regulatory reporting. This is accomplished through the synergistic integration of embedding-based vectorization, similarity computation, threshold-driven mapping validation, and persistent graph-based storage of semantic relationships, resulting in measurable improvements to both system efficiency and data quality.

The data input to any ML model and/or the data output from any ML model, as described herein, may be used for operations performed by one or more of the following: Database Software, Cloud Infrastructure Software, Customer Relationship Management Software, Data Science Software, Digital Assistant Software, Vision Software, Language Software, Speech Software, Forecasting Software, Enterprise Software, Middleware, Server Software, Identity Management Software, Application Development Software, Analytics Software, Security Software, Data Integration Software, Health Software, Hospitality Software, Retail Software, Utilities Software, Operating Systems, Virtualization Software, Governance and Administration Software, Migration & Disaster Recovery Software, Networking Software, Connectivity Software, Monitoring Software, Procurement Software, Project Management Software, Risk Management Software, Supply Chain Management Software, Manufacturing Software, Human Capital Management Software, Customer Experience Software, Advertising Software, and Industry-Specific Application Software.

### 9. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

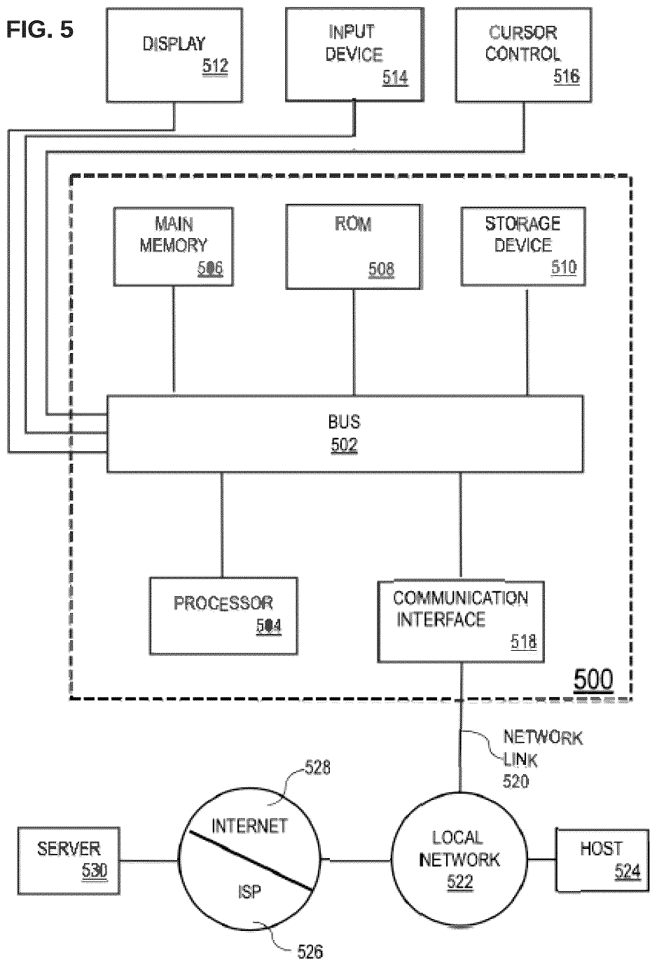

For example, FIG. 5 is a block diagram that illustrates a computer system 500 upon which an embodiment of the disclosure may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk, optical disk, or a Solid State Drive (SSD) is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### 10. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected, and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
accessing a plurality of sets of terminology,
wherein each set of terminology, in the plurality of sets of terminology, comprises a respective plurality of terms;
generating a cross-terminology network that comprises:
(a) a plurality of nodes, each of the plurality of nodes representing at least one term in at least one set of terminology of the plurality of sets of terminology;
(b) an inter-terminology connection between:
(i) a first node, of the plurality of nodes, that represents a first term in a first set of terminology of the plurality of sets of terminology; and
(ii) a second node, of the plurality of nodes, that represents a second term in a second set of terminology of the plurality of sets of terminology; and,
(c) an intra-terminology connection between:
(i) a third node, of the plurality of nodes, that represents a third term in the first set of terminology; and
(ii) a fourth node, of the plurality of nodes, that represents a fourth term in the first set of terminology;
identifying a first set of relationships for the first node representing the first term to a first subset of nodes in the cross-terminology network that respectively represent a first set of terms;
identifying a second set of relationships for a fifth node representing a fifth term in the second set of terminology to a second subset of nodes in the cross-terminology network that respectively represent a second set of terms;
applying a vector embedding function to the first set of relationships and/or the first set of terms to generate a first vector embedding for the first term;
applying the vector embedding function to the second set of relationships and/or the second set of terms to generate a second vector embedding for the fifth term;
computing a first similarity measure based on the first vector embedding and the second vector embedding; and
based at least on the first similarity measure, mapping the fifth term in the second set of terminology as a semantic match of the first term in the first set of terminology.

2. The one or more non-transitory computer readable media of claim 1, wherein the operations further comprise:
identifying a third set of relationships for a sixth node representing a sixth term to a third subset of nodes in the cross-terminology network that respectively represent a third set of terms;
applying the vector embedding function to the third set of relationships and/or the third set of terms to generate a third vector embedding for the sixth term;
computing a second similarity measure based on the first vector embedding and the third vector embedding; and
based at least on the second similarity measure, refraining from mapping the sixth term in the second set of terminology as a semantic match of the first term in the first set of terminology.

3. The one or more non-transitory computer readable media of claim 1, wherein the first similarity measure meets a threshold for mapping the second term in the second set of terminology as a semantic match of the first term in the first set of terminology.

4. The one or more non-transitory computer readable media of claim 1, wherein the first set of terminology includes a set of standard codes and the second set of terminology includes a set of proprietary codes.

5. The one or more non-transitory computer readable media of claim 1, wherein the first similarity measure comprises a cosine similarity measure for the first vector embedding and the second vector embedding.

6. The one or more non-transitory computer readable media of claim 1, wherein the operations further comprise:
applying a reasoner to terms within the plurality of sets of terminologies to determine a first relationship between the first term and the second term;
wherein generating the cross-terminology network comprises generating the inter-terminology connection based on the first relationship determined by the reasoner.

7. The one or more non-transitory computer readable media of claim 6, wherein the operations further comprise:
applying the reasoner to terms within the plurality of sets of terminologies to determine a second relationship between the third term and the fourth term;
wherein generating the cross-terminology network comprises generating the intra-terminology connection based on the second relationship determined by the reasoner.

8. The one or more non-transitory computer readable media of claim 1, wherein the operations further comprise:
generating a diagnostic determination for the patient based on the mapped standard code, the diagnostic determination comprising an identification of a target condition and a confidence score.

9. The one or more non-transitory computer readable media of claim 1, wherein the operations further comprise:
initiating a treatment order set based on the mapped standard code, the treatment order set comprising at least one of: (i) a medication order, (ii) a laboratory order, (iii) an imaging order, or (iv) a nursing protocol.

10. A method comprising:
accessing a plurality of sets of terminology,
wherein each set of terminology, in the plurality of sets of terminology, comprises a respective plurality of terms;
generating a cross-terminology network that comprises:
(d) a plurality of nodes, each of the plurality of nodes representing at least one term in at least one set of terminology of the plurality of sets of terminology;
(e) an inter-terminology connection between:
(iii) a first node, of the plurality of nodes, that represents a first term in a first set of terminology of the plurality of sets of terminology; and
(iv) a second node, of the plurality of nodes, that represents a second term in a second set of terminology of the plurality of sets of terminology;
(f) an intra-terminology connection between:
(iii) a third node, of the plurality of nodes, that represents a third term in the first set of terminology; and
(iv) a fourth node, of the plurality of nodes, that represents a fourth term in the first set of terminology;
identifying a first set of relationships for the first node representing the first term to a first subset of nodes in the cross-terminology network that respectively represent a first set of terms;
identifying a second set of relationships for a fifth node representing a fifth term in the second set of terminology to a second subset of nodes in the cross-terminology network that respectively represent a second set of terms;
applying a vector embedding function to the first set of relationships and/or the first set of terms to generate a first vector embedding for the first term;
applying the vector embedding function to the second set of relationships and/or the second set of terms to generate a second vector embedding for the fifth term;
computing a first similarity measure based on the first vector embedding and the second vector embedding; and
based at least on the first similarity measure, mapping the fifth term in the second set of
terminology as a semantic match of the first term in the first set of terminology, wherein the method is performed by at least one device including a hardware processor.

11. The method of claim 10, further comprising:
identifying a third set of relationships for a sixth node representing a sixth term to a third subset of nodes in the cross-terminology network that respectively represent a third set of terms;
applying the vector embedding function to the third set of relationships and/or the third set of terms to generate a third vector embedding for the sixth term;
computing a second similarity measure based on the first vector embedding and the third vector embedding; and
based at least on the second similarity measure, refraining from mapping the sixth term in the second set of terminology as a semantic match of the first term in the first set of terminology.

12. The method of claim 10, wherein the first similarity measure meets a threshold for mapping the second term in the second set of terminology as a semantic match of the first term in the first set of terminology.

13. The method of claim 10, wherein the first set of terminology includes a set of standard codes and the second set of terminology includes a set of proprietary codes.

14. The method of claim 10, wherein the first similarity measure comprises a cosine similarity measure for the first vector embedding and the second vector embedding.

15. The method of claim 10, further comprising:
applying a reasoner to terms within the plurality of sets of terminologies to determine a first relationship between the first term and the second term;
wherein generating the cross-terminology network comprises generating the inter-terminology connection based on the first relationship determined by the reasoner.

16. The method of claim 15, further comprising:
applying the reasoner to terms within the plurality of sets of terminologies to determine a second relationship between the third term and the fourth term;
wherein generating the cross-terminology network comprises generating the intra-terminology connection based on the second relationship determined by the reasoner.

17. A system comprising:
one or more hardware processors;
one or more non-transitory computer readable media; and
program instructions stored on the one or more non-transitory computer readable media which, when executed by the one or more hardware processors, cause the system to perform operations comprising:
accessing a plurality of sets of terminology,
wherein each set of terminology, in the plurality of sets of terminology, comprises a respective plurality of terms;
generating a cross-terminology network that comprises:
(g) a plurality of nodes, each of the plurality of nodes representing at least one term in at least one set of terminology of the plurality of sets of terminology;
(h) an inter-terminology connection between:
(v) a first node, of the plurality of nodes, that represents a first term in a first set of terminology of the plurality of sets of terminology; and
(vi) a second node, of the plurality of nodes, that represents a second term in a second set of terminology of the plurality of sets of terminology;
(i) an intra-terminology connection between:
(v) a third node, of the plurality of nodes, that represents a third term in the first set of terminology; and
(vi) a fourth node, of the plurality of nodes, that represents a fourth term in the first set of terminology;
identifying a first set of relationships for the first node representing the first term to a first subset of nodes in the cross-terminology network that respectively represent a first set of terms;
identifying a second set of relationships for a fifth node representing a fifth term in the second set of terminology to a second subset of nodes in the cross-terminology network that respectively represent a second set of terms;
applying a vector embedding function to the first set of relationships and/or the first set of terms to generate a first vector embedding for the first term;
applying the vector embedding function to the second set of relationships and/or the second set of terms to generate a second vector embedding for the fifth term;
computing a first similarity measure based on the first vector embedding and the second vector embedding; and
based at least on the first similarity measure, mapping the fifth term in the second set of terminology as a semantic match of the first term in the first set of terminology.

18. The system of claim 17, wherein the operations further comprise:
identifying a third set of relationships for a sixth node representing a sixth term to a third subset of nodes in the cross-terminology network that respectively represent a third set of terms;
applying the vector embedding function to the third set of relationships and/or the third set of terms to generate a third vector embedding for the sixth term;
computing a second similarity measure based on the first vector embedding and the third vector embedding; and
based at least on the second similarity measure, refraining from mapping the sixth term in the second set of terminology as a semantic match of the first term in the first set of terminology.

19. The system of claim 17, wherein the first similarity measure meets a threshold for mapping the second term in the second set of terminology as a semantic match of the first term in the first set of terminology.

20. The system of claim 17, wherein the operations further comprise:
applying a reasoner to terms within the plurality of sets of terminologies to determine a first relationship between the first term and the second term;
wherein generating the cross-terminology network comprises generating the inter-terminology connection based on the first relationship determined by the reasoner; and
applying the reasoner to terms within the plurality of sets of terminologies to determine a second relationship between the third term and the fourth term,
wherein generating the cross-terminology network comprises generating the intra-terminology connection based on the second relationship determined by the reasoner.
